(19) United States
(12) Patent Application Publication
(10) Pub. No.: US 2006/0019932 A1
(43) Pub. Date: Jan. 26, 2006

(54) Treatment of rheumatoid arthritis by inhibition of pde4

(76) Inventors: Joseph Anthony Mancini, Kirkland (CA); David Claveau, Laval (CA); Chi Chung Chan, Kirkland (CA); Donald Nicholson, Montreal (CA); Denise M Visco, Fanwood, NJ (US)

Correspondence Address: MERCK AND CO., INC, P O BOX 2000, RAHWAY, NJ, 07065-0907, US

(21) Appl. No.: 10/526,346

(22) PCT Filed: Sep. 2, 2003
(86) PCT No.: PCT/US03/27612

§ 371 Date: Mar. 2, 2005

Related U.S. Application Data
(60) Provisional application No. 60/408,597, filed on Sep. 6, 2002.

(51) Int. Cl. A61K 31/655; A61K 31/44; A61K 31/4172; A61K 31/47

(52) U.S. Cl. 514/151; 514/311; 514/357; 514/396

(57) ABSTRACT

A method of treatment of rheumatoid arthritis administers an effective amount of a compound that inhibits phospho-diesterase-4.

### BACKGROUND OF THE INVENTION

1. Field of the Invention

The present invention is directed to a method of treatment of rheumatoid arthritis by administering an effective amount of a compound that is a phosphodiesterase-4 inhibitor.

2. Related Background

Hormones are compounds that variously affect cellular activity. In many respects, hormones act as messengers to trigger specific cellular responses and activities. Many effects produced by hormones, however, are not caused by the singular effect of just the hormone. Instead, the hormone first binds to a receptor, thereby triggering the release of a second compound that goes on to affect the cellular activity. In this scenario, the hormone is known as the first messenger while the second compound is called the second messenger. Cyclic adenosine monophosphate (adenosine 3′,5′-cyclic monophosphate, “cAMP” or “cyclic AMP”) is known as a second messenger for hormones including epinephrine, glucagon, calcitonin, corticotrophin, lipotropin, luteinizing hormone, norepinephrine, parathyroid hormone, thyroid-stimulating hormone, and vasopressin. Thus, cAMP mediates cellular responses to hormones. Cyclic AMP also mediates cellular responses to various neurotransmitters.

Phosphodiesterases (“PDE”) are a family of enzymes that metabolize 3′,5′ cyclic nucleotides to 5′ nucleoside monophosphates, thereby terminating cAMP second messenger activity. A particular phosphodiesterase, phosphodiesterase-4 (“PDE4”, also known as “PDE-IV”), which is a high affinity, cAMP specific, type IV PDE, has generated interest as potential targets for the development of novel anti-asthmatic and anti-inflammatory compounds. PDE4 is known to exist as at lease four isoenzymes, each of which is encoded by a distinct gene. Each of the four known PDE4 gene products is believed to play varying roles in allergic and/or inflammatory responses. Thus, it is believed that inhibition of PDE4, particularly the specific PDE4 isoforms that produce detrimental responses, can beneficially affect allergy and inflammation symptoms. It would be desirable to provide a method of treatment of rheumatoid arthritis by administering compounds and compositions that inhibit PDE4 activity.

Inhibition of PDE4 activity is believed effective for the treatment of osteoporosis by reducing bone loss. For example, Ken-ici Miyamoto et al., Biochem. Pharmacology, 54: 613-617(1997) describes the effect of a PDE4 on bone loss. A major concern with the use of PDE4 inhibitors is the side effect of emesis which has been observed for several candidate compounds as described in C. Burnouf et al., (“Burnouf”), Ann. Rep. In Med. Chem., 33: 91-109(1998). B. Hughes et al., Br. J. Pharmacol., 118: 1183-1191(1996); M. J. Perry et al., Cell Biochem. Biophys., 29: 113-132(1998); S. B. Christensen et al., J. Med. Chem., 41: 821-835(1998); and Burnouf describe the wide variation of the severity of the undesirable side effects exhibited by various compounds. As described in M. D. Houslay et al., Adv. In Pharmacol., 44: 225-342(1998) and D. Spina et al., Adv. In Pharmacol., 44: 33-89(1998), there is great interest and research of therapeutic PDE4 inhibitors.

U.S. Pat. Nos. 5,622,977, 5,710,160, 5,710,170, 5,798,373, 5,849,770, and International Patent Publication No. WO 99/50262 describe tri-substituted aryl derivative PDE IV inhibitors, including tri-aryl ethane derivatives. U.S. Pat. No. 6,410,563 describes 8-arylquinoline compounds that are PDE4 inhibitors. U.S. Pat. No. 5,712,298 describes other PDE4 inhibitors.

Compounds that include ringed systems are described by various investigators as effective for a variety of therapies and utilities. For example, International Patent Publication No. WO 98/25883 describes ketobenzamides as calpain inhibitors, European Patent Publication No. EP 811610 and U.S. Pat. Nos. 5,679,712, 5,693,672 and 5,747,541 describe substituted benzoylguanidine sodium channel blockers, U.S. Pat. No. 5,736,297 describes ring systems useful as a photosensitive composition. International Patent Publication WO9422852 describes quinolines as PDE4 inhibitors.

U.S. Pat. Nos. 5,491,147, 5,608,070, 5,739,144, 5,776,958, 5,780,477, 5,786,354, 5,859,034, 5,866,593, 5,891,896, and International Patent Publication WO 95/35283 describe PDE4 inhibitors that are tri-substituted aryl or heteroaryl phenyl derivatives. U.S. Pat. No. 5,580,888 describes PDE4 inhibitors that are styryl derivatives. U.S. Pat. No. 5,550,137 describes PDE4 inhibitors that are phenylaminocarbonyl derivatives. U.S. Pat. No. 5,340,827 describes PDE4 inhibitors that are phenylcarboxamide compounds. U.S. Pat. No. 5,780,478 describes PDE4 inhibitors that are tetra-substituted phenyl derivatives. International Patent Publication WO 96/00215 describes substituted oxime derivatives useful as PDE4 inhibitors. U.S. Pat. No. 5,633,257 describes PDE4 inhibitors that are cyclo(alkyl and alkenyl)phenyl-alkenyl (aryl and heteroaryl) compounds.

However, there remains a need for a method of treatment of rheumatoid arthritis with minimal side effects.

### BRIEF DESCRIPTION OF THE DRAWINGS

FIG. 1 is a graphical plot of the cumulative score in collagen-induced arthritis in DBA mice plotted against days dosed for vehicle, Roflumilast, Indomethacin, and Example 1.

### SUMMARY OF THE INVENTION

The present invention provides a method of treatment in mammals of rheumatoid arthritis by the administration of an effective amount of a phosphodiesterase-4 inhibitor.

### DETAILED DESCRIPTION OF THE INVENTION

Rheumatoid arthritis (RA) is a systemic autoimmune disease resulting in progressive joint destruction and associated pain. Three major contributors to its pathology are joint inflammation, abnormal cellular and humoral responses, and synovial hyperplasia. T-lymphocytes (T-cells) and monocytes are the major cell types recovered from the synovial tissue of RA patients and experimental evidence confirms that pro-inflammatory cytokines produced by these cells are the major contributors to joint pathogenesis. The inventors have analyzed human whole blood for the production of various cytokines including Th1 and Th2 cytokines following stimulation with lipoplysaccharide (LPS) and Concanavalin A (ConA) in the presence or absence of PDE4 inhibitors. These studies demonstrated that the PDE4 inhibitors decreased the formation of GM-CSF, IFN-γ, IL-2, IL-12, and TNF-α. IFNγ is primarily a Th1 response and inhibition in whole blood and isolate human T cells suggest that PDE4 inhibitors predominantly inhibit Th1 cytokines. TNF-α sequestrants alone have shown clinical efficacy in RA. Thus, if sufficient levels of inhibition are achieved, PDE4 inhibitor-mediated TNF-α suppression would have efficacy in RA.

In one aspect, the present invention provides a method of treatment of rheumatoid arthritis by administering to one in need of such treatment an effective amount of a compound that inhibits PDE4.

In a second aspect, the present invention provides a method of treatment of rheumatoid arthritis by administering to one in need of such treatment an effective amount of a compound represented by Formula (I): 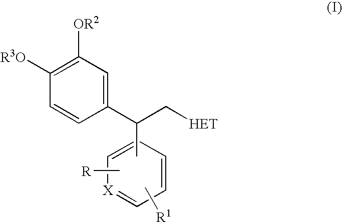
or a pharmaceutically acceptable salt thereof wherein:

R is hydrogen, C₁₋₆alkyl, halogen or CF₃;

R¹ is —(CH₂)ₘ—CO—N(R⁴)—S(O)₂—R⁵, —(CH₂)ₘ—CO—N(R⁴)—S(O)₂—NR⁶R⁷, —(CH₂)ₘ—S(O)₂—N(R⁴)—CO—R⁴, —(CH₂)ₘ—S(O)₂—N(R⁴)—CO—NR⁶R⁷, or —C(OH)(C₁₋₆haloalkyl)₂, wherein m is 0, 1 or 2,

R² and R³ are each independently C₁₋₇alkyl, substituted C1-7 alkyl, wherein the substituent is F, Cl, Br or I, 2-phenethyl or 2-indanyl, optionally mono or di-substituted, wherein the substituents on the benzene ring are each independently halogen, —C₁₋₆alkoxy, —C₁₋₆alkylthio, —CN, —CF₃, —C₁₋₆alkyl, —N₃, or —CO₂H.

R⁴ is hydrogen, —C₁₋₆alkyl, phenyl, benzyl or 2-phenethyl, optionally mono or di-substituted, wherein the substituents on the benzene ring are independently halo, —C₁₋₆alkoxy, —C₁₋₆alkylthio, —CN, —CF₃, —C₁₋₆alkyl, —N₃, or —CO₂H.

R⁵, R⁸ and R¹¹ are each independently —CF₃, —C₁₋₆alkyl, phenyl, benzyl or 2-phenethyl, optionally mono or di-substituted, wherein the substituents on the benzene ring are independently halogen, —C₁₋₆alkoxy, —C₁₋₆alkylthio, —CN, —CF₃, —C₁₋₆alkyl, —N₃, or —CO₂H.

R⁶, R⁷, R⁹ and R¹⁰ are each independently hydrogen, or —C₁₋₆alkyl, or

R⁶ and R⁷ may be joined to form a saturated 5, 6 or 7 membered heterocycle, said heterocycle containing a heteroatom which is nitrogen and optionally containing an additional hetero atom which is an O or an S atom or NR⁴, and optionally containing a carbonyl group;

HET is pyridyl or imidazolyl, optionally mono-, or disubstituted, wherein the substituents are independently halogen, —C₁₋₆alkyl, —C₁₋₆alkoxy, —C₁₋₆alkylthio, benzyl, 2-phenethyl, —NHCOR⁸, —NR⁹R¹⁰, —NHS(O)₂R¹¹, OH, —CN, or —CF₃, or the N-oxides thereof; and
X is N, N→O, or CH

Compounds represented by Formula (I) are described in U.S. Pat. No. 5,710,170.

In a third aspect, this invention provides a method of treatment of rheumatoid arthritis by administering to one in need of such treatment an effective amount of a compound represented by Formula (II): 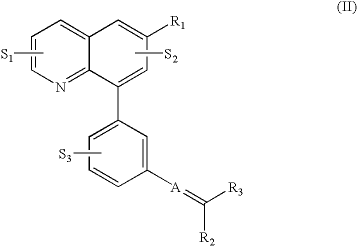
or a pharmaceutically acceptable salt thereof, wherein

S₁, S₂, and S₃ are independently H, —OH, halogen, —C₁-C₆alkyl, —NO₂, —CN, or —C₁-C₆alkoxy, wherein the alkyl and alkoxy groups are optionally substituted with 1-5 substituents; wherein each substituent is independently a halogen or OH;

R₁ is a H, OH, halogen, or —C₁-C₆alkyl, -cycloC₃-C₆alkyl, —C₁-C₆alkenyl, —C₁-C₆alkoxy, aryl, heteroaryl, —CN, -heterocycloC₃-C₆alkyl, -amino, —C₁-C₆alkylamino, —(C₁-C₆alkyl)(C₁-C₆alkyl)amino, —C₁-C₆alkyl(oxy)C₁-C₆alkyl, —C(O)NH(aryl), —C(O)NH(heteroaryl), —SOₙNH(aryl), —SOₙNH(heteroaryl), —SOₙNH(C₁-C₆alkyl), —C(O)N(C₀-C₆alkyl)(C₀-C₆alkyl), —NH—SOₙ—(C₁-C₆alkyl), —SOₙ—(C₁-C₆alkyl), —(C₁-C₆alkyl)—O—C(CN)-dialkylamino, or —(C₁-C₆alkyl)—SOₙ—(C₁-C₆alkyl) group, wherein any of the groups is optionally substituted with 1-5 substituents; wherein each substituent is independently a halogen, —OH, —CN, —C₁-C₆alkyl, -cycloC₃-C₆alkyl, —C(O)(heterocycloC₃-C₆alkyl), —C(O)—O—(C₀-C₆alkyl), —C(O)-aryloxy, —C₁-C₆alkoxy, —(C₀-C₆alkyl)(C₀-C₆alkyl)amino, cycloalkyloxy, acyl, acyloxy, -cycloC₃-C₆alkyl, heterocycloC₃-C₆alkyl, aryl, heteroaryl, carbamoyl, or —SOₙ—(C₁-C₆alkyl);

A is CH, C-ester, or C—R₄;

R₂ and R₃ independently is an aryl, heteroaryl, H, halogen, —CN, —C₁-C₆alkyl, heterocycloC₃₋₆alkyl, —C₁-C₆alkoxy, carbamoyl, —C(O)OH, —(C₁-C₆alkyl)—SOₙ—(C₁-C₆alkyl), —C(O)N(C₀-C₆alkyl)(C₀-C₆alkyl), or —C₁-C₆alkylacylamino group, wherein any of the groups is optionally substituted with 1-5 substituents, wherein each substituent is independently an aryl, heteroaryl, halogen, —NO₂, —C(O)OH, —CN, —C₁-C₆alkyl, —SOₙ—(C₁-C₆alkyl), —SOₙ-(aryl), aryloxy, -heteroaryloxy, C₁-C₆alkoxy, N-oxide, —C(O)-heterocycloC₃-C₆alkyl, —NH-cycloC₃-C₆alkyl, amino, —OH, or —(C₀-C₆alkyl)(C₀-C₆alkyl)amino, —C(O)—N(C₀-C₆alkyl)(C₀-C₆alkyl) substituent group, wherein each substituent group independently is optionally substituted with —OH, C₁-C₆alkoxy, —C₁-C₆alkyl, -cycloC₃-C₆alkyl, aryloxy, —C(O)OH, —C(O)O(C₁-C₆alkyl), halogen, —NO₂, —CN, —SOₙ—(C₁-C₆alkyl), or —C(O)—N(C₀-C₆alkyl)(C₀-C₆alkyl);

one of R₂ and R₃ must be an aryl or heteroaryl, optionally substituted;

when R₂ and R₃ are both an aryl or heteroaryl, then R₂ and R₃ may be optionally connected by a thio, oxy, or (C₁-C₄alkyl) bridge to form a fused three ring system;

R₄ is an aryl, —C₁-C₆alkyl, heteroaryl, —CN, carbamoyl, —(C₁-C₆alkyl)—SOₙ—(C₁-C₆alkyl), —C(O)N(C₀-C₆alkyl)(C₀-C₆alkyl), or —C₁-C₆alkylacylamino group, wherein any of the groups is optionally substituted with 1-5 substituents, wherein each substituent is independently a —CN, halogen, —C(O)(C₀-C₆alkyl), —C(O)O(C₀-C₆alkyl), —C₁-C₆alkyl, —SOₙ—(C₁-C₆alkyl), —OH, C₁-C₆alkoxy, or —(C₀-C₆alkyl)(C₀-C₆alkyl)amino, group;

n is independently 0, 1, or 2; and

R₂ or R₃ may optionally be joined to R₄ by a bond to form a ring.

Compounds represented by Formulat (II) are described in U.S. Pat. No. 6,410,563.

In a fourth aspect, the invention provides a method of treating rheumatoid arthritis by administering to one in need of such treatment an effective A compound represented by Formula (M): 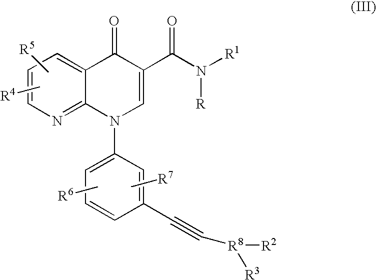
or a pharmaceutically acceptable salt thereof, wherein

R is H, —C₁₋₆alkyl or —C₃₋₆cycloalkyl;

R¹ is H, or a —C₁₋₆alkyl, —C₃₋₆cycloalkyl, —C₁₋₆alkoxy, —C₂₋₆alkenyl, —C₃₋₆alkynyl, —C(O)C₁₋₆alkyl, —C(O)-aryl, —(C₀₋₆alkyl)—SOₙ—(C₁₋₆alkyl), —(C₀₋₆alkyl)—SOₙ-(aryl), phenyl, heteroaryl, or heterocycloC₃₋₇alkyl group, wherein any of the groups is optionally substituted with 1-3 independent —C₁₋₆alkyl, —C₁₋₆alkoxy, OH, —N(C₀₋₆alkyl)(C₀₋₆alkyl), —(C₀₋₆alkyl)—SOₙ—(C₁₋₆alkyl), nitro, CN, ═N—O—C₁₋₆alkyl, —O—N═C₁₋₆alkyl, or halogen substituents;

R² is absent, H, halogen, —C₁₋₆alkyl, —C₃₋₆cycloalkyl, —C₁₋₆alkyl(C₃₋₆cycloalkyl)(C₃₋₆cycloalkyl), —C₁₋₆alkoxy, phenyl, heteroaryl, heterocycloC₃₋₇alkyl, nitro, CN, ═N—O—C₁₋₆alkyl, —O—N═C₁₋₆alkyl, —N(C₀₋₆alkyl)(C₀₋₆alkyl), —NHSOₙ—(C₁₋₆alkyl), —NHC(O)—C₁₋₆alkyl, —NHC(O)-aryl, —C(O)—C₁₋₆alkyl, —C(O)—O—C₁₋₆alkyl, —C₁₋₆alkyl(═N—OH), —C(N═NOH)C₁₋₆alkyl, —C₀₋₆alkyl(oxy)C₁₋₆alkyl-phenyl, —SOₙNH(C₀₋₆alkyl), or —(C₀₋₆alkyl)—SOₙ—(C₁₋₆alkyl), wherein the phenyl, heteroaryl or heterocycloC₃₋₇alkyl is optionally substituted with halogen, —C₁₋₆alkyl, —C₁₋₆alkoxy, hydroxy, —N(C₀₋₆alkyl)(C₀₋₆alkyl), or —C(O)—O—C₁₋₆alkyl, and any alkyl is optionally substituted with 1-6 independent halogen or —OH substituents;

n is 0, 1, or 2;

R³ is absent, H, OH, —N(C₀₋₆alkyl)(C₀₋₆alkyl), halogen or C₁₋₆alkyl, wherein any alkyl is optionally substituted with 1-6 independent halogen, OH, or —N(C₀₋₆alkyl)(C₀₋₆alkyl) substituents;

R⁴, R⁵, R⁶, and R⁷ each independently is H, halogen, —C₁₋₆alkyl, —C₁₋₆alkoxy, —SOₙ—(C₁₋₆alkyl), nitro, CN, or —N(C₀₋₆alkyl)(C₀₋₆alkyl), and any alkyl is optionally substituted with 1-6 independent halogen or —OH substituents; and

R⁸ is phenyl, pyridyl, pyrimidyl, indolyl, quinolinyl, thienyl, pyridonyl, oxazolyl, oxadiazolyl, thiazolyl, thiadiazolyl, or imidazolyl; or oxides thereof when R⁸ is a heteroaryl; or H, —C₁₋₆alkyl, or —C₃₋₆cycloalkyl, and any alkyl is optionally substituted with 1-6 independent halogen, —N(C₀₋₆alkyl)(C₀₋₆alkyl), —N(C₃₋₇cycloalkyl)(C₀₋₆alkyl), —N(C₃₋₇cycloalkyl)(C₃₋₇cycloalkyl), N-heterocycloC₄₋₇alkyl, —SOₙ—(C₁₋₆alkyl), —SOₙ-(aryl), or —OH substituents.

The compounds represented by formula (III) are described in PCT publication WO 03/018579. Specific compounds of Formula III disclosed in WO 03/018579 are as follows:

TABLE 1

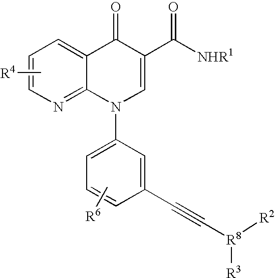

| Ex. | R¹ | R⁸R²R³ | R⁴ | R⁶ |
|---|---|---|---|---|
| 1 | i-pr | Ph | H | H |
| 2 | i-pr | 2-Pyr | H | H |
| 3 | i-pr | 4-Pyr | H | H |
| 4 | i-pr | 4-Pyr NO | H | H |
| 5 | i-pr | H | H | H |
| 6 | c-pr | H | H | H |
| 7 | i-pr | 3-Pyr | H | H |
| 8 | i-pr | 3-Pyr NO | H | H |
| 9 | c-pr | 3-Pyr | H | H |
| 10 | i-pr | 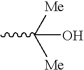 | H | H |
| 11 | c-pr | 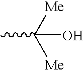 | H | H |
| 12 | i-pr | 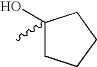 | H | H |
| 13 | i-pr | 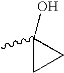 | H | H |
| 14 | i-pr | 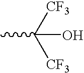 | H | H |
| 15 | i-pr | 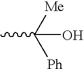 | H | H |
| 16 | c-pr | 3-Pyr NO | H | H |
| 17 | i-pr | 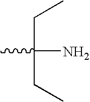 | H | H |
| 18 | c-pr | 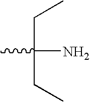 | H | H |
| 19 | i-pr | 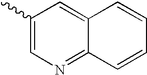 | H | H |
| 20 | i-pr | 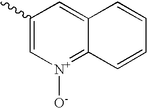 | H | H |
| 21 | i-pr | 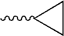 | H | H |
| 22 | i-pr | 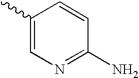 | H | H |
| 23 | i-pr | 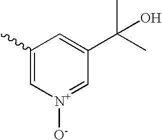 | H | H |
| 24 | i-pr | 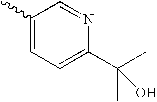 | H | H |
| 25 | i-pr | 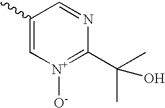 | H | H |
| 26 | i-pr | 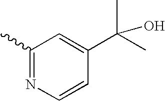 | H | H |
| 27 | i-pr | 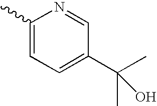 | H | H |
| 28 | i-pr | 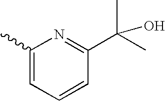 | H | H |
| 29 | c-pr | 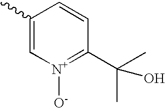 | H | H |
| 30 | i-pr | 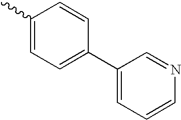 | H | H |
| 31 | i-pr | 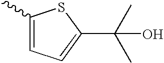 | H | H |
| 32 | i-pr | 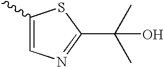 | H | H |
| 33 | H | 3-Pyr NO | H | H |

TABLE 2

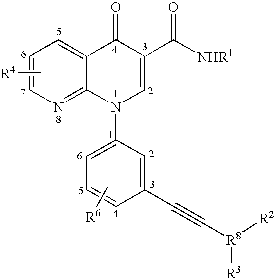

| Ex. | R¹ | R⁸R²R³ | R⁴ | R⁶ |
|---|---|---|---|---|
| 35 | 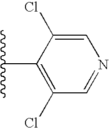 | 3-Pyr-NO | H | H |
| 36 | c-Bu | 3-(OH)Ph | 6-Me | H |
| 37 | 3-Pyr | Ph | H | 5-Me |
| 38 | CH₂Ph | 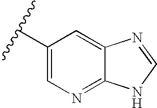 | H | H |
| 39 | 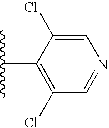 | 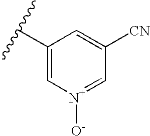 | H | H |
| 40 | 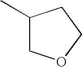 | 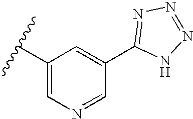 | 7-Me | H |
| 41 | Ph | 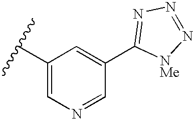 | 6-Cl | H |
| 42 | 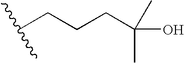 | 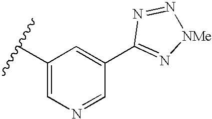 | H | 4-Cl |
| 43 | 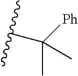 | 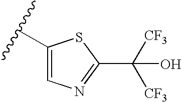 | H | H |
| 44 | 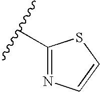 | 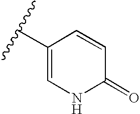 | H | 5-OH |
| 45 | 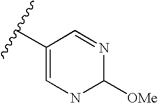 | 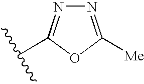 | H | H |
| 46 | 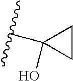 | 3-Pyr-NO | H | H |
| 47 | 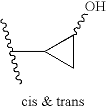 | 3-Pyr-NO | H | H |
| 48 | 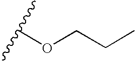 | 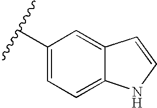 | H | H |
| 49 | SO₂Ph | 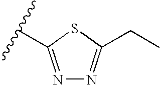 | H | H |
| 50 | 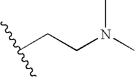 | 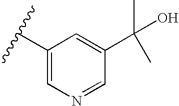 | H | 5-F |
| 51 | 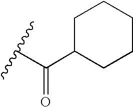 | 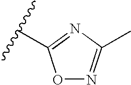 | 5-OH | H |
| 52 | Me | 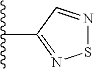 | H | H |
| 53 | Et | 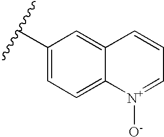 | H | H |

As used herein, “alkyl” as well as other groups having the prefix “alk” such as, for example, alkoxy, alkanoyl, alkenyl, alkynyl and the like, means carbon chains which may be linear or branched or combinations thereof. Examples of alkyl groups include methyl, ethyl, propyl, isopropyl, butyl, sec- and tert-butyl, pentyl, hexyl, heptyl and the like. “Alkenyl”, “alkynyl” and other like terms include carbon chains containing at least one unsaturated C—C bond.

The term “cycloalkyl” means carbocycles containing no heteroatoms, and includes mono-, bi- and tricyclic saturated carbocycles, as well as fused ring systems. Such fused ring systems can include one ring that is partially or fully unsaturated such as a benzene ring to form fused ring systems such as benzofused carbocycles. Cycloalkyl includes such fused ring systems as spirofused ring systems. Examples of cycloalkyl include cyclopropyl, cyclobutyl, cyclopentyl, cyclohexyl, decahydronaphthalenyl, adamantanyl, indanyl, indenyl, fluorenyl, 1,2,3,4-tetrahydronaphthalenyl and the like. Similarly, “cycloalkenyl” means carbocycles containing no heteroatoms and at least one non-aromatic C—C double bond, and include mono-, bi- and tricyclic partially saturated carbocycles, as well as benzofused cycloalkenes. Examples of cycloalkenyl include cyclohexenyl, indenyl, and the like.

The term “cycloalkyloxy” unless specifically stated otherwise includes a cycloalkyl group connected to the oxy connecting atom.

The term “alkoxy” unless specifically stated otherwise includes an alkyl group connected to the oxy connecting atom.

The term “aryl” unless specifically stated otherwise includes multiple ring systems as well as single ring systems such as, for example, phenyl or naphthyl.

The term “aryloxy” unless specifically stated otherwise includes multiple ring systems as well as single ring systems such as, for example, phenyl or naphthyl, connected through the oxy connecting atom to the connecting site.

The term “C₀-C₆alkyl” includes alkyls containing 6, 5, 4, 3, 2, 1, or no carbon atoms. An alkyl with no carbon atoms is a hydrogen atom substituent when the alkyl is a terminus moiety. An alkyl with no carbon atoms is a direct bond when the alkyl is a bridging moiety.

The term “hetero” unless specifically stated otherwise includes one or more O, S, or N atoms. For example, heterocycloalkyl and heteroaryl include ring systems that contain one or more O, S, or N atoms in the ring, including mixtures of such atoms. The heteroatoms replace ring carbon atoms. Thus, for example, a heterocycloC₅alkyl is a five membered ring containing from 5 to no carbon atoms.

Examples of heteroaryl include, for example, pyridinyl, quinolinyl, isoquinolinyl, pyridazinyl, pyrimidinyl, pyrazinyl, quinoxalinyl, furyl, benzofuryl, dibenzofuryl, thienyl, benzothienyl, pyrrolyl, indolyl, pyrazolyl, indazolyl, oxazolyl, isoxazolyl, thiazolyl, isothiazolyl, imidazolyl, benzimidazolyl, oxadiazolyl, thiadiazolyl, triazolyl, tetrazolyl.

The term “heteroaryloxy” unless specifically stated otherwise describes a heteroaryl group connected through an oxy connecting atom to the connecting site.

Examples of heteroaryl(C₁₋₆)alkyl include, for example, furylmethyl, furylethyl, thienylmethyl, thienylethyl, pyrazolylmethyl, oxazolylmethyl, oxazolylethyl, isoxazolylmethyl, thiazolylmethyl, thiazolylethyl, imidazolylmethyl, imidazolylethyl, benzimidazolylmethyl, oxadiazolylmethyl, oxadiazolylethyl, thiadiazolylmethyl, thiadiazolylethyl, triazolylmethyl, triazolylethyl, tetrazolylmethyl, tetrazolylethyl, pyridinylmethyl, pyridinylethyl, pyridazinylmethyl, pyrimidinylmethyl, pyrazinylmethyl, quinolinylmethyl, isoquinolinylmethyl and quinoxalinylmethyl.

Examples of heterocycloC₃₋₇alkyl include, for example, azetidinyl, pyrrolidinyl, piperidinyl, perhydroazepinyl, piperazinyl, morpholinyl, tetrahydrofuranyl, imidazolinyl, pyrolidin-2-one, piperidin-2-one, and thiomorpholinyl.

The term “N-heterocycloC₄₋₇alkyl” describes nonaryl heterocyclic compounds having 3-6 carbon atoms and one nitrogen atom forming the ring. Examples include azetidinyl, pyrrolidinyl, piperidinyl, and perhydroazepinyl.

Examples of aryl(C₁₋₆)alkyl include, for example, phenyl(C₁₋₆)alkyl, and naphthyl(C₁₋₆)alkyl.

Examples of heterocycloC₃₋₇alkylcarbonyl(C₁₋₆)alkyl include, for example, azetidinyl carbonyl(C₁₋₆)alkyl, pyrrolidinyl carbonyl(C₁₋₆)alkyl, piperidinyl carbonyl(C₁₋₆)alkyl, piperazinyl carbonyl(C₁₋₆)alkyl, morpholinyl carbonyl(C₁₋₆)alkyl, and thiomorpholinyl carbonyl(C₁₋₆)alkyl.

The term “amine” unless specifically stated otherwise includes primary, secondary and tertiary amines substituted with C₁-C₄alkyl.

Unless otherwise stated, the term “carbamoyl” is used to include —NHC(O)OC₁-C₄alkyl, and —OC(O)NHC₁-C₄alkyl.

The term “halogen” includes fluorine, chlorine, bromine and iodine atoms.

The term “optionally substituted” is intended to include both substituted and unsubstituted. Thus, for example, optionally substituted aryl could represent a pentafluorophenyl or a phenyl ring. Further, the substitution can be made at any of the groups. For example, substituted aryl(C₁₋₆)alkyl includes substitution on the aryl group as well as substitution on the alkyl group.

The term “oxide” of heteroaryl groups is used in the ordinary well-known chemical sense and include, for example, N-oxides of nitrogen heteroatoms.

Compounds described herein contain one or more double bonds and may thus give rise to cis/trans isomers as well as other conformational isomers. The present invention includes all such possible isomers as well as mixtures of such isomers.

Compounds described herein can contain one or more asymmetric centers and may thus give rise to diastereomers and optical isomers. The present invention includes all such possible diastereomers as well as their racemic mixtures, their substantially pure resolved enantiomers, all possible geometric isomers, and pharmaceutically acceptable salts thereof. The above Formula I and Formula II are shown without a definitive stereochemistry at certain positions. The present invention includes all stereoisomers of Formula I and Formula II and pharmaceutically acceptable salts thereof. Further, mixtures of stereoisomers as well as isolated specific stereoisomers are also included. During the course of the synthetic procedures used to prepare such compounds, or in using racemization or epimerization procedures known to those skilled in the art, the products of such procedures can be mixtures of stereoisomers.

The term “pharmaceutically acceptable salts” refers to salts prepared from pharmaceutically acceptable non-toxic bases or acids. When the compound of the present invention is acidic, its corresponding salt can be conveniently prepared from pharmaceutically acceptable non-toxic bases, including inorganic bases and organic bases. Salts derived from such inorganic bases include aluminum, ammonium, calcium, copper (ic and ous), ferric, ferrous, lithium, magnesium, manganese (ic and ous), potassium, sodium, zinc and the like salts. Particularly preferred are the ammonium, calcium, magnesium, potassium and sodium salts. Salts derived from pharmaceutically acceptable organic non-toxic bases include salts of primary, secondary, and tertiary amines, as well as cyclic amines and substituted amines such as naturally occurring and synthesized substituted amines. Other pharmaceutically acceptable organic non-toxic bases from which salts can be formed include ion exchange resins such as, for example, arginine, betaine, caffeine, choline, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyarrine resins, procaine, purines, theobromine, triethylamine, trimethylamine, tripropylamine, tromethamine and the like.

When the compound of the present invention is basic, its corresponding salt can be conveniently prepared from pharmaceutically acceptable non-toxic acids, including inorganic and organic acids. Such acids include, for example, acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid and the like. Particularly preferred are benzenesulfonic, citric, hydrobromic, hydrochloric, maleic, phosphoric, sulfuric, and tartaric acids.

The pharmaceutical compositions utilized by the present invention comprise a compound represented by Formula I or Formula II (or pharmaceutically acceptable salts thereof) as an active ingredient, a pharmaceutically acceptable carrier and optionally other therapeutic ingredients or adjuvants. Such additional therapeutic ingredients include, for example, i) Leukotriene receptor antagonists, ii) Leukotriene biosynthesis inhibitors, iii) corticosteroids, iv) H1 receptor antagonists, v) beta 2 adrenoceptor agonists, vi) COX-2 selective inhibitors, vii) statins, viii) non-steroidal anti-inflammatory drugs (“NSAID”), and ix) M2/M3 antagonists. The compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions may be conveniently presented in unit dosage form and prepared by any of the methods well known in the art of pharmacy.

Creams, ointments, jellies, solutions, or suspensions containing the compound of Formula I can be employed for topical use. Mouth washes and gargles are included within the scope of topical use for the purposes of this invention.

Dosage levels from about 0.001 mg/kg to about 140 mg/kg of body weight per day are useful in the treatment of rheumatoid arthritis, or alternatively about 0.05 mg to about 7 g per patient per day. Further, it is understood that the PDE4 inhibiting compounds can be administered at prophylactically effective dosage levels to prevent the above-recited conditions.

The amount of active ingredient that may be combined with the carrier materials to produce a single dosage form will vary depending upon the host treated and the particular mode of administration. For example, a formulation intended for the oral administration to humans may conveniently contain from about 0.5 mg to about 5 g of active agent, compounded with an appropriate and convenient amount of carrier material which may vary from about 5 to about 95 percent of the total composition. Unit dosage forms will generally contain between from about 0.01 mg to about 1000 mg of the active ingredient, typically 0.01 mg, 0.05 mg, 0.25 mg, 1 mg, 5 mg, 25 mg, 50 mg, 100 mg, 200 mg, 300 mg, 400 mg, 500 mg, 600 mg, 800 mg or 1000 mg.

It is understood, however, that the specific dose level for any particular patient will depend upon a variety of factors including the age, body weight, general health, sex, diet, time of administration, route of administration, rate of excretion, drug combination and the severity of the particular disease undergoing therapy.

In practice, the compounds represented by Formula I or Formula II, or pharmaceutically acceptable salts thereof, of this invention can be combined as the active ingredient in intimate admixture with a pharmaceutical carrier according to conventional pharmaceutical compounding techniques. The carrier may take a wide variety of forms depending on the form of preparation desired for administration, e.g., oral or parenteral (including intravenous). Thus, the pharmaceutical compositions of the present invention can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion or as a water-in-oil liquid emulsion. In addition to the common dosage forms set out above, the compound represented by Formula I, or pharmaceutically acceptable salts thereof, may also be administered by controlled release means and/or delivery devices. The compositions may be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

Thus, the pharmaceutical compositions of this invention may include a pharmaceutically acceptable carrier and a compound or a pharmaceutically acceptable salt of Formula I or Formula II. The compounds of Formula I or Formula II, or pharmaceutically acceptable salts thereof, can also be included in pharmaceutical compositions in combination with one or more other therapeutically active compounds.

The pharmaceutical carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

In preparing the compositions for oral dosage form, any convenient pharmaceutical media may be employed. For example, water, glycols, oils, alcohols, flavoring agents, preservatives, coloring agents and the like may be used to form oral liquid preparations such as suspensions, elixirs and solutions; while carriers such as starches, sugars, microcrystalline cellulose, diluents, granulating agents, lubricants, binders, disintegrating agents, and the like may be used to form oral solid preparations such as powders, capsules and tablets. Because of their ease of administration, tablets and capsules are the preferred oral dosage units whereby solid pharmaceutical carriers are employed. Optionally, tablets may be coated by standard aqueous or nonaqueous techniques.

A tablet containing the composition of this invention may be prepared by compression or molding, optionally with one or more accessory ingredients or adjuvants. Compressed tablets may be prepared by compressing, in a suitable machine, the active ingredient in a free-flowing form such as powder or granules, optionally mixed with a binder, lubricant, inert diluent, surface active or dispersing agent. Molded tablets may be made by molding in a suitable machine, a mixture of the powdered compound moistened with an inert liquid diluent. Each tablet preferably contains from about 0.1 mg to about 500 mg of the active ingredient and each cachet or capsule preferably containing from about 0.1 mg to about 500 mg of the active ingredient.

Pharmaceutical compositions of the present invention suitable for parenteral administration may be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present invention suitable for injectable use include sterile aqueous solutions or dispersions. Furthermore, the compositions can be in the form of sterile powders for the extemporaneous preparation of such sterile injectable solutions or dispersions. In all cases, the final injectable form must be sterile and must be effectively fluid for easy syringability. The pharmaceutical compositions must be stable under the conditions of manufacture and storage; thus, preferably should be preserved against the contaminating action of microorganisms such as bacteria and fungi. The carrier can be a solvent or dispersion medium containing, for example, water, ethanol, polyol (e.g. glycerol, propylene glycol and liquid polyethylene glycol), vegetable oils, and suitable mixtures thereof.

Pharmaceutical compositions of the present invention can be in a form suitable for topical use such as, for example, an aerosol, cream, ointment, lotion, dusting powder, or the like. Further, the compositions can be in a form suitable for use in transdermal devices. These formulations may be prepared, utilizing a compound represented by Formula I or Formula II, or pharmaceutically acceptable salts thereof, via conventional processing methods. As an example, a cream or ointment is prepared by mixing hydrophilic material and water, together with about 5 wt % to about 10 wt % of the compound, to produce a cream or ointment having a desired consistency.

Pharmaceutical compositions of this invention can be in a form suitable for rectal administration wherein the carrier is a solid. It is preferable that the mixture forms unit dose suppositories. Suitable carriers include cocoa butter and other materials commonly used in the art. The suppositories may be conveniently formed by first admixing the composition with the softened or melted carrier(s) followed by chilling and shaping in moulds.

In addition to the aforementioned carrier ingredients, the pharmaceutical formulations described above may include, as appropriate, one or more additional carrier ingredients such as diluents, buffers, flavoring agents, binders, surface-active agents, thickeners, lubricants, preservatives (including anti-oxidants) and the like. Furthermore, other adjuvants can be included to render the formulation isotonic with the blood of the intended recipient. Compositions containing a compound described by Formula I or Formula II, or pharmaceutically acceptable salts thereof, may also be prepared in powder or liquid concentrate form.

Further, as described above, the compound of this invention can be utilized in combination with other therapeutic compounds. In particular, the combinations of the PDE4 inhibiting compound of this invention can be advantageously used in combination with i) Leukotriene receptor antagonists, ii) Leukotriene biosynthesis inhibitors, iii) COX-2 selective inhibitors, iv) statins, v) NSAIDs, vi) M2/M3 antagonists, vii) corticosteroids, viii) H1 (histamine) receptor antagonists and ix) beta 2 adrenoceptor agonist.

Thus, for example, rheumatoid arthritis can be conveniently treated with capsules, cachets or tablets each containing 1 mg, 5 mg, 25 mg, 50 mg, 100 mg, 200 mg, 300 mg, 400 mg, or 500 mg of the active ingredient of the compound of the present application, or a pharmaceutically acceptable salt thereof, administered once, twice, or three times daily.

### Assays Demonstrating Biological Activity LPS and FMLP-Induced TNF-α and LTB₄ Assays in Human Whole Blood

Whole blood provides a protein and cell-rich milieu appropriate for the study of biochemical efficacy of anti-inflammatory compounds such as PDE4-selective inhibitors. Normal non-stimulated human blood does not contain detectable levels of TNF-α and LTB_{4.} Upon stimulation with LPS, activated monocytes express and secrete TNF-α up to 8 hours and plasma levels remain stable for 24 hours. Published studies have shown that inhibition of TNF-α by increasing intracellular cAMP via PDE4 inhibition and/or enhanced adenylyl cyclase activity occurs at the transcriptional level. LTB₄ synthesis is also sensitive to levels of intracellular cAMP and can be completely inhibited by PDE4-selective inhibitors. As there is little LTB₄ produced during a 24 hour LPS stimulation of whole blood, an additional LPS stimulation followed by fMLP challenge of human whole blood is necessary for LTB₄ synthesis by activated neutrophils. Thus, by using the same blood sample, it is possible to evaluate the potency of a compound on two surrogate markers of PDE4 activity in the whole blood by the following procedure.

Fresh blood was collected in heparinized tubes by venipuncture from healthy human volunteers (male and female). These subjects had no apparent inflammatory conditions and had not taken any NSAIDs for at least 4 days prior to blood collection. 500 μL aliquots of blood were pre-incubated with either 2 μL of vehicle (DMSO) or 2 μL of test compound at varying concentrations for 15 minutes at 37° C. This was followed by the addition of either 10 μL vehicle (PBS) as blanks or 10 μL LPS (1 μg/mL final concentration, #L-2630 (Sigma Chemical Co., St. Louis, Mo.) from E. coli, serotype 011:B4; diluted in 0.1% w/v BSA (in PBS)). After 24 hours of incubation at 37° C., another 10 μL of PBS (blank) or 10 μL of LPS (1 μg/mL final concentration) was added to blood and incubated for 30 minutes at 37° C. The blood was then challenged with either 10 μL of PBS (blank) or 10 μL of fMLP (1 μM final concentration, #F-3506 (Sigma); diluted in 1% w/v BSA (in PBS)) for 15 minutes at 37° C. The blood samples were centrifuged at 1500 xg for 10 minutes at 4° C. to obtain plasma. A 50 μL aliquot of plasma was mixed with 200 μL methanol for protein precipitation and centrifuged as above. The supernatant was assayed for LTB₄ using an enzyme immunoassay kit (#520111 from Cayman Chemical Co., Ann Arbor, Mich.) according to the manufacturer's procedure. TNF-α was assayed in diluted plasma (in PBS) using an ELISA kit (Cistron Biotechnology, Pine Brook, N.J.) according to manufacturer's procedure. IC₅₀ values should be less than about 5 μM, advantageously less than about 2.5 μM.

### Anti-Allergic Activity in Vivo

Compounds of the invention have been tested for effects on an IgE-mediated allergic pulmonary inflammation induced by inhalation of antigen by sensitized guinea pigs. Guinea pigs were initially sensitized to ovalbumin under mild cyclophosphamide-induced immunosuppression, by intraperitoneal injection of antigen in combinations with aluminum hydroxide and pertussis vaccine. Booster doses of antigen were given two and four weeks later. At six weeks, animals were challenged with aerosolized ovalbumin while under cover of an intraperitoneally administered anti-histamine agent (mepyramine). After a further 48 h, bronchial alveolar lavages (BAL) were performed and the numbers of eosinophils and other leukocytes in the BAL fluids were counted. The lungs were also removed for histological examination for inflammatory damage. Administration of compounds of the Examples (0.001-10 mg/kg i.p. or p.o.), up to three times during the 48 h following antigen challenge, lead to a significant reduction in the eosinophilia and the accumulation of other inflammatory leukocytes.

### Spa Based PDE Activity Assay Protocol

Compounds which inhibit the hydrolysis of cAMP to AMP by the type-IV cAMP-specific phosphodiesterases were screened in a 96-well plate format as follows:

In a 96 well-plate at 30° C. the test compound was added (dissolved in 2 μL DMSO), 188 μL of substrate buffer containing [2,8-³H] adenosine 3′,5′-cyclic phosphate (cAMP, 100 nM to 50 μM), 10 mM MgCl₂, 1 mM EDTA, 50 mM Tris, pH 7.5. The reaction was initiated by the addition of human recombinant PDE4 (the amount was controlled so that ˜10% product was formed in 10 min.). The reaction was stopped after 10 min. by the addition of 1 mg of PDE-SPA beads (Amersham Pharmacia Biotech, Inc., Piscataway, N.J.). The product AMP generated was quantified on a Wallac Microbeta® 96-well plate counter (EG&G Wallac Co., Gaithersburg, Md.). The signal in the absence of enzyme was defined as the background. 100% activity was defined as the signal detected in the presence of enzyme and DMSO with the background subtracted. Percentage of inhibition was calculated accordingly. IC₅₀ value was approximated with a non-linear regression fit using the standard 4-parameter/multiple binding sites equation from a ten point titration.

The IC₅₀ values of Examples 1 and 2 were determined with 100 nM cAMP using the purified GST fusion protein of the human recombinant phosphodiesterase IVa (met-248) produced from a baculovirus/Sf-9 expression system. IC₅₀ values should be less than about 1000 nM, advantageously less than about 250 nM, and even more advantageously less than about 100 nM. The IC₅₀ values of Examples 1 and 2 were 0.76 nM and 1.31 nM respectively.

### EXAMPLE 1

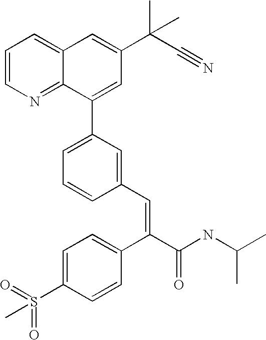

EXAMPLE 1 can be prepared as described in U.S. Pat. No. 6,410,563.

### EXAMPLE 2

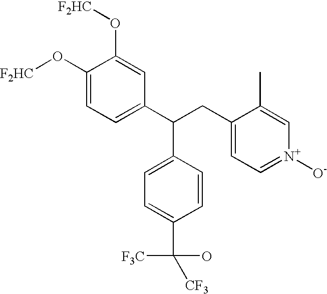

EXAMPLE 2 can be prepared as described in U.S. Pat. No. 5,710,170.

### EXAMPLE 3

EXAMPLE 3 is Roflumilast, N-(3,5-dichloropyrid-4-yl)-3-cyclopropylmethoxy-4-difluoromethoxybenzamide, described in U.S. Pat. No. 5,712,298.

Inhibition of various Th1 and monocyte derived cytokines which may play a role in human rheumatoid arthritis.

Inhibition of LPS or Con A stimulate human whole blood μM

| Compound | IL-2 | IFNγ | TNF-α | IL-12 | GM-CSF |
|---|---|---|---|---|---|
| EX. 2 | 0.3 | 0.05 | 0.05 | 0.13 | 0.07 |

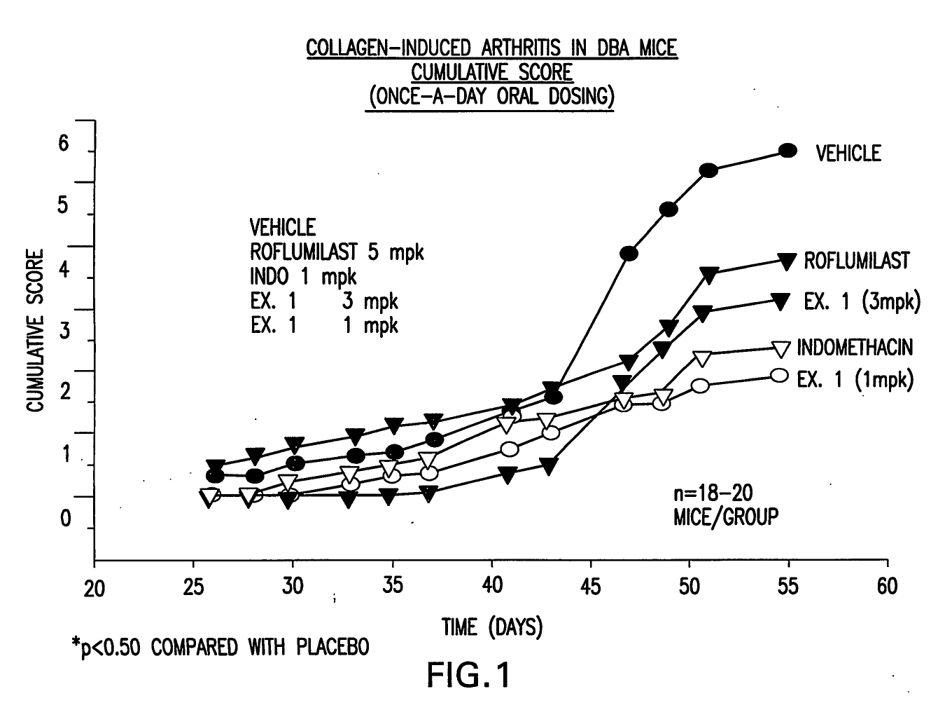

These results, together with the low cumulative scores shown in FIG. 1, comparable to indomethacin, shows that the PDE4 inhibiting compounds EXAMPLE 1, EXAMPLE 2, and Roflumilast, may be effective to treat rheumatoid arthritis.

1. A method of treatment of rheumatoid arthritis by administering, to one in need of such treatment, an effective amount of a phosphodiesterase-4 inhibiting compound.

2. A method of treatment of rheumatoid arthritis according to claim 1 by administering, to one in need of such treatment, an effective amount of a compound represented by Formula (I): 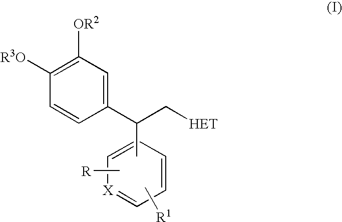
or a pharmaceutically acceptable salt thereof wherein:
R is hydrogen, C₁₋₆alkyl, halogen or CF₃;
R¹ is —(CH₂)ₘ—CO—N(R⁴)—S(O)₂—R⁵, —(CH₂)ₘ—CO—N(R⁴)—S(O)₂—NR⁶R⁷, —(CH₂)ₘ—S(O)₂—N(R⁴)—CO—R⁴, —(CH₂)ₘ—S(O)₂—N(R⁴)—CO—NR⁶R⁷, or —C(OH)(C₁₋₆haloalkyl)₂, wherein m is 0, 1 or 2.
R² and R³ are each independently C₁₋₇alkyl, substituted C₁₋₇ alkyl, wherein the substituent is F, Cl, Br or I, 2-phenethyl or 2-indanyl, optionally mono or di-substituted, wherein the substituents on the benzene ring are each independently halogen, —C₁₋₆alkoxy, —C₁₋₆alkylthio, —CN, —CF₃, —C₁₋₆alkyl, —N₃, or —CO₂H.
R⁴ is hydrogen, —C₁₋₆alkyl, phenyl, benzyl or 2-phenethyl, optionally mono or di-substituted, wherein the substituents on the benzene ring are independently halo, —C₁₋₆alkoxy, —C₁₋₆alkylthio, —CN, —CF₃, —C₁₋₆alkyl, —N₃, or —CO₂H.
R⁵, R⁸ and R¹¹ are each independently —CF₃, —C₁₋₆alkyl, phenyl, benzyl or 2-phenethyl, optionally mono or di-substituted, wherein the substituents on the benzene ring are independently halogen, —C₁₋₆alkoxy, —C₁₋₆alkylthio, —CN, —CF₃, —C₁₋₆alkyl, N₃, or CO₂H.
R⁶, R⁷, R⁹ and R¹⁰ are each independently hydrogen, or —C₁₋₆alkyl, or
R⁶ and R⁷ may be joined to form a saturated 5, 6 or 7 membered heterocycle, said heterocycle containing a heteroatom which is nitrogen and optionally containing an additional hetero atom which is an O or an S atom or NR⁴, and optionally containing a carbonyl group;
HET is pyridyl or imidazolyl, optionally mono-, or disubstituted, wherein the substituents are independently halogen, —C₁₋₆alkyl, —C₁₋₆alkoxy, —C₁₋₆alkylthio, benzyl, 2-phenethyl, —NHCOR⁸, —NR⁹R¹⁰, —NHS(O)₂R¹¹, OH, —CN, or —CF₃, or the N-oxides thereof; and
X is N, N→O, or CH

3. A method of treatment of rheumatoid arthritis according to claim 1 by administering to one in need of such treatment an effective amount of a compound represented by Formula (II): 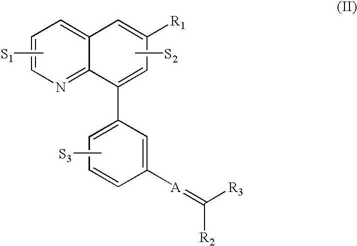
or a pharmaceutically acceptable salt thereof, wherein
S₁, S₂, and S₃ are independently H, —OH, halogen, —C₁-C₆alkyl, —NO₂, —CN, or —C₁-C₆alkoxy, wherein the alkyl and alkoxy groups are optionally substituted with 1-5 substituents; wherein each substituent is independently a halogen or OH;
R₁ is a H, OH, halogen, or —C₁-C₆alkyl, -cycloC₃-C₆alkyl, —C₁-C₆alkenyl, —C₁-C₆alkoxy, aryl, heteroaryl, —CN, -heterocycloC₃-C₆alkyl, -amino, —C₁-C₆alkylamino, —(C₁-C₆alkyl)(C₁-C₆alkyl)amino, —C₁-C₆alkyl(oxy)C₁-C₆alkyl, —C(O)NH(aryl), —C(O)NH(heteroaryl), —SOₙNH(aryl), —SOₙNH(heteroaryl), —SOₙNH(C₁-C₆alkyl), —C(O)N(C₀-C₆alkyl)(C₀-C₆alkyl), —NH—SOₙ—(C₁-C₆alkyl), —SOₙ—(C₁-C₆alkyl), —(C₁-C₆alkyl)—O—C(CN)-dialkylamino, or —(C₁-C₆alkyl)—SOₙ—(C₁-C₆alkyl) group, wherein any of the groups is optionally substituted with 1-5 substituents; wherein each substituent is independently a halogen, —OH, —CN, —C₁-C₆alkyl, -cycloC₃-C₆alkyl, —C(O)(heterocycloC₃-C₆alkyl), —C(O)—O—(C₀-C₆alkyl), —C(O)-aryloxy, —C₁-C₆alkoxy, —(C₀-C₆alkyl)(C₀-C₆alkyl)amino, cycloalkyloxy, acyl, acyloxy, -cycloC₃-C₆alkyl, heterocycloC₃-C₆alkyl, aryl, heteroaryl, carbamoyl, or —SOₙ—(C₁-C₆alkyl);
A is CH, C-ester, or C—R₄;
R₂ and R₃ independently is an aryl, heteroaryl, H, halogen, —CN, —C₁-C₆alkyl, heterocycloC₃₋₆alkyl, —C₁-C₆alkoxy, carbamoyl, —C(O)OH, —(C₁-C₆alkyl)—SOₙ—(C₁-C₆alkyl), —C(O)N(C₀-C₆alkyl)(C₀-C₆alkyl), or —C₁-C₆alkylacylamino group, wherein any of the groups is optionally substituted with 1-5 substituents, wherein each substituent is independently an aryl, heteroaryl, halogen, —NO₂, —C(O)OH, —CN, —C₁-C₆alkyl, —SOₙ—(C₁-C₆alkyl), —SOₙ—(aryl), aryloxy, -heteroaryloxy, C₁-C₆alkoxy, N-oxide, —C(O)-heterocycloC₃-C₆alkyl, —NH-cycloC₃-C₆alkyl, amino, —OH, or —(C₀-C₆alkyl)(C₀-C₆alkyl)amino, —C(O)—N(C₀-C₆alkyl)(C₀-C₆alkyl) substituent group, wherein each substituent group independently is optionally substituted with —OH, C₁-C₆alkoxy, —C₁-C₆alkyl, -cycloC₃-C₆alkyl, aryloxy, —C(O)OH, —C(O)O(C₁-C₆alkyl), halogen, —NO₂, —CN, —SOₙ—(C₁-C₆alkyl), or —C(O)—N(C₀-C₆alkyl)(C₀-C₆alkyl);
one of R₂ and R₃ must be an aryl or heteroaryl, optionally substituted;
when R₂ and R₃ are both an aryl or heteroaryl, then R₂ and R₃ may be optionally connected by a thio, oxy, or (C₁-C₄alkyl) bridge to form a fused three ring system;
R₄ is an aryl, —C₁-C₆alkyl, heteroaryl, —CN, carbamoyl, —(C₁-C₆alkyl)—SOₙ—(C₁-C₆alkyl), —C(O)N(C₀-C₆alkyl)(C₀-C₆alkyl), or —C₁-C₆alkylacylamino group, wherein any of the groups is optionally substituted with 1-5 substituents, wherein each substituent is independently a —CN, halogen, —C(O)(C₀-C₆alkyl), —C(O)O(C₀-C₆alkyl), —C₁-C₆alkyl, —SOₙ—(C₁-C₆alkyl), —OH, C₁-C₆alkoxy, or —(C₀-C₆alkyl)(C₀-C₆alkyl)amino, group;
n is independently 0, 1, or 2; and
R₂ or R₃ may optionally be joined to R₄ by a bond to form a ring.

4. The method of claim 2, wherein said compound is represented by

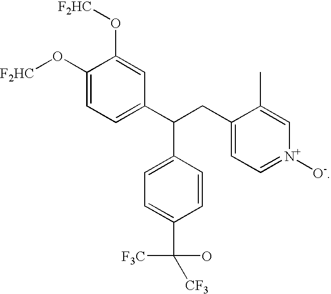

5. The method of claim 3, wherein said compound is represented by

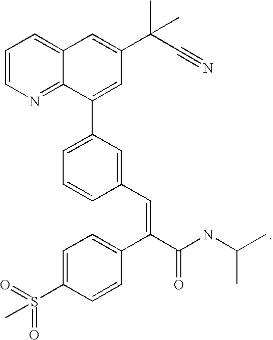

6. A method of treatment of rheumatoid arthritis by administering to one in need of such treatment an effective amount of N-(3,5-dichloropyrid-4-yl)-3-cyclopropylmethoxy-4-difluoromethoxybenzamide.

7. A method of treatment of rheumatoid arthritis by administering, to one in need of such treatment, an effective amount of a compound represented by Formula (IE): 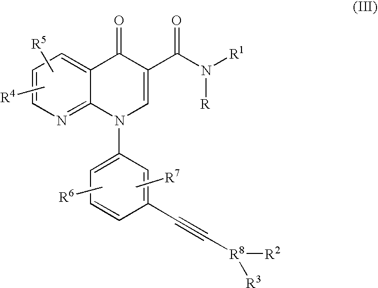
or a pharmaceutically acceptable salt thereof, wherein
R is H, —C₁₋₆alkyl or —C₃₋₆cycloalkyl;
R¹ is H, or a —C₁₋₆alkyl, —C₃₋₆cycloalkyl, —C₁₋₆alkoxy, —C₂₋₆alkenyl, —C₃₋₆alkynyl, —C(O)—C₁₋₆alkyl, —C(O)-aryl, —(C₀₋₆alkyl)—SOₙ—(C₁₋₆alkyl), —(C₀₋₆alkyl)—SOₙ-(aryl), phenyl, heteroaryl, or heterocycloC₃₋₇alkyl group, wherein any of the groups is optionally substituted with 1-3 independent —C₁₋₆alkyl, —C₁₋₆alkoxy, OH, —N(C₀₋₆alkyl)(C₀₋₆alkyl), —(C₀₋₆alkyl)—SOₙ—(C₁₋₆alkyl), nitro, CN, ═N—O—C₁₋₆alkyl, —O—N═C₁₋₆alkyl, or halogen substituents;
R² is absent, H, halogen, —C₁₋₆alkyl, —C₃₋₆cycloalkyl, —C₁₋₆alkyl(C₃₋₆cycloalkyl)(C₃₋₆cycloalkyl), —C₁₋₆alkoxy, phenyl, heteroaryl, heterocycloC₃₋₇alkyl, nitro, CN, ═N—O—C₁₋₆alkyl, —O—N═C₁₋₆alkyl, —N(C₀₋₆alkyl)(C₀₋₆alkyl), —NHSOₙ—(C₁₋₆alkyl), —NHC(O)—C₁₋₆alkyl, —NHC(O)-aryl, —C(O)—C₁₋₆alkyl, —C(O)—O—C₁₋₆alkyl, —C₁₋₆alkyl(═N—OH), —C(N═NOH)C₁₋₆alkyl, —C₀₋₆alkyl(oxy)C₁₋₆alkyl-phenyl, —SOₙNH(C₀₋₆alkyl), or —(C₀₋₆alkyl)—SOₙ—(C₁₋₆alkyl), wherein the phenyl, heteroaryl or heterocycloC₃₋₇alkyl is optionally substituted with halogen, —C₁₋₆alkyl, —C₁₋₆alkoxy, hydroxy, —N(C₀₋₆alkyl)(C₀₋₆alkyl), or —C(O)—O—C₁₋₆alkyl, and any alkyl is optionally substituted with 1-6 independent halogen or —OH substituents;
n is 0, 1,or 2;
R³ is absent, H, OH, —N(C₀₋₆alkyl)(C₀₋₆alkyl), halogen or C₁₋₆alkyl, wherein any alkyl is optionally substituted with 1-6 independent halogen, OH, or —N(C₀₋₆alkyl)(C₀₋₆alkyl) substituents;
R⁴, R⁵, R⁶, and R⁷ each independently is H, halogen, —C₁₋₆alkyl, —C₁₋₆alkoxy, —SOₙ—(C₁₋₆alkyl), nitro, CN, or —N(C₀₋₆alkyl)(C₀₋₆alkyl), and any alkyl is optionally substituted with 1-6 independent halogen or —OH substituents; and
R⁸ is phenyl, pyridyl, pyrimidyl, indolyl, quinolinyl, thienyl, pyridonyl, oxazolyl, oxadiazolyl, thiazolyl, thiadiazolyl, or imidazolyl; or oxides thereof when R⁸ is a heteroaryl; or H, —C₁₋₆alkyl, or —C₃₋₆cycloalkyl, and any alkyl is optionally substituted with 1-6 independent halogen, —N(C₀₋₆alkyl)(C₀₋₆alkyl), —N(C₃₋₇cycloalkyl)(C₀₋₆alkyl), —N(C₃₋₇cycloalkyl)(C₃₋₇cycloalkyl), N-heterocycloC₄₋₇alkyl, —SOₙ—(C₁₋₆alkyl), —SOₙ-(aryl), or —OH substituents.

8. A compound according to claim 7 wherein
R is hydrogen;
R¹ is cyclopropyl;
R, R⁴ R⁵, R⁶ and R⁷ are each hydrogen; and
R⁸(R²)(R³) is 3-pyridine N-oxide.
